(19) United States
(12) Patent Application Publication
(10) Pub. No.: US 2012/0071662 A1
(43) Pub. Date: Mar. 22, 2012

(54) PREPARING AMINOARYLALKYL COMPOUNDS

(75) Inventors: Michael Sander, Rosrath (DE); Wolfgang Stirner, Bergisch Gladbach (DE); Frank Laschinski, Leverkusen (DE); Michael Konrad, Leverkusen (DE); Karsten Von Dem Bruch, Leverkusen (DE)

(73) Assignee: SALTIGO GMBH, Langenfeld (DE)

(21) Appl. No.: 13/197,046

(22) Filed: Aug. 3, 2011

(30) Foreign Application Priority Data
Aug. 6, 2010 (US) 10 2010 033 690.4

(51) Int. Cl. C07D 213/73; C07D 213/75

(52) U.S. Cl. 546/309; 546/311

(57) ABSTRACT

The invention relates to a process for preparing aminoarylalkyl compounds, more particularly 5-amino-2-isopropylpyridine.

The invention relates to a process for preparing aminoarylalkyl compounds, more particularly 5-amino-2-isopropylpyridine.

Aminoarylalkyl compounds, more particularly 5-amino-2-isopropylpyridine, are useful intermediates for synthesizing medicinal products. EP 1852431 A describes (1S)-(−)-N-[(1-ethyl-1H-pyrazol-4-yl)methyl]-5-hydroxy-N-(6-isopropylpyridin-3-yl)-1,2,3,4-tetrahydronaphthalene-1-carboxamide as the prophylactic or/and therapeutic anti-inflammatory and preparing it from 5-amino-2-isopropylpyridine. 5-Amino-2-isopropylpyridine is obtainable from an N-protected aminoarylalkyl compound, viz. 5-(N-(Boc)-amino)-2-isopropylpyridine, by elimination of the protective group.

EP 1852431 A further discloses that 5-amino-2-isopropylpyridine is obtainable from 2-hydroxy-6-isopropylnicotinonitrile. JP 2008-222593 A describes a process for preparing 5-amino-2-isopropylpyridine from 2-isopropylpyridine-5-carboxamide by Hofmann degradation using sodium hypochlorite.

The existing processes for preparing anainoarylalkyl compounds, more particularly 5-amino-2-isopropylpyridine, all have in common that they are not efficiently implementable on an industrial scale and that their yield is too low given the number of process steps.

There accordingly continues to be a need for a process for preparing aminoarylalkyl compounds which overcomes the disadvantages of the prior art and provides aminoarylalkyl compounds in good yields and efficiently on an industrial scale.

Surprisingly, aminoarylalkyl compounds were found to be convertible into N-protected aminoarylalkyl compounds in good yields starting from N,N-protected aminoarylalkylhalogen compounds and iron-catalysed coupling with Grignard compounds. These N-protected aminoarylalkyl compounds can then be converted into the atninoarylalkyl compound by elimination of the protective group.

The invention accordingly provides a process for preparing the compounds of formula (1)

R¹—NH-ARYL-R² (1)

where R¹ is —COOR³ or —SO₂—R⁴, where R³ and R⁴ are each selected from the group: C₁-C₁₀-alkyl, C₂-C₁₀-alkenyl, C₁-C₁₀-perhaloalkyl, C₇-C₁₅-arylalkyl or C₆-C₂₄-aryl, or R³ or R⁴ is C₁-C₁₀-alkyl or C₆-C₂₄-aryl each singly or multiply but not wholly substituted by C₁-C₁₀-alkoxy, Cl, Br or F, or R¹ is —SO₂—NH—(C₁-C₁₀-alkyl), —SO₂—NH—(C₇-C₁₅-arylalkyl), —SO₂NH—(C₆-C₂₄-aryl) or SO₂(NR⁵R⁶), where R⁵ and R⁶ each represent C₁-C₁₀-alkyl, or NR⁵R⁶ together form a 5- to 7-membered ring, and
ARYL represents a substituted or unsubstituted carbocyclic C₆-C₂₄-aryl radical or a substituted or unsubstituted heteroaromatic C₃-C₁₆-hetaryl radical, and
R² is C₁-C₁₀-alkyl, C₂-C₁₀-alkenyl, C₆-C₂₄-aryl, C₇-C₁₅-arylalkyl, C₃-C₁₆-hetaryl or a 3- to 7-membered saturated or partially unsaturated heterocycle which may optionally be further substituted by radicals selected from the group: C₁-C₁₀-alkyl, C₂-C₁₀-alkenyl, C₁-C₁₀-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-aryl alkyl), —OCOO—(C₁-C₁₀-alkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₁-C₁₀-alkyl), —CO—(C₆-C₂₄-aryl), —SO₂—NH—(C₁-C₁₀-alkyl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring, in that compounds of formula (2)

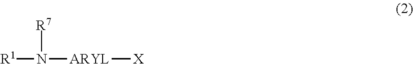

where R¹ and R⁷ may be the same or different and R¹ and ARYL are each as defined above and R⁷ is —COOR³ or —SO₂—R⁴, where R³ and R⁴ are each selected from the group: C₁-C₁₀-alkyl, C₂-C₁₀-alkenyl, C₁-C₁₀-perhaloalkyl, C₆-C₂₄-aryl or C₇-C₁₅-arylalkyl or R³ or R⁴ is C₁-C₁₀-alkyl or C₆-C₂₄-aryl each singly or multiply but not wholly substituted by C₁-C₁₀-alkoxy, Cl, Br or F, or R¹ is —SO₂—NH—(C₁-C₁₀-alkyl), —SO₂—NH—(C₇-C₁₅-arylalkyl), —SO₂NH—(C₆-C₂₄-aryl) or —SO₂(NR⁵R⁶), where R⁵ and R⁶ each represent C₁-C₁₀-alkyl, or NR⁵R⁶ together form a 5- to 7-membered ring, and
X is Cl, Br, I or —OSO₂—R⁸ where R⁸ is C₁-C₁₀-alkyl, C₁-C₁₀-perhaloalkyl, C₇-C₁₅-arylalkyl or C₆-C₇₄-aryl or R⁸ is C₁-C₁₀-alkyl or C₆-C₂₄-aryl each singly or multiply but not wholly substituted by C₁-C₁₀-alkoxy, Cl, Br or F, are reacted in the presence of at least one iron source with compounds of formula (3)

R²-MeY (3)

where R² is as defined above and Y is an anionic ligand and Me is a metal selected from the group Mg, Ca, Mn, Zn, to form compounds of formula (1).

ARYL preferably represents unsubstituted C₆-C₂₄-aryl or unsubstituted C₃-C₁₆-hetaryl or C₆-C₂₄-aryl substituted by one to three identical or different substituents or C₃-C₁₆-hetaryl substituted by one to three identical or different substituents, wherein the substituents are selected from the group: C₁-C₁₀-alkyl, C₂-C₁₀-alkenyl, C₁-C₁₀-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), SO₂(C₆-C₂₄-aryl), —CO—(C₁-C₁₀-alkyl), —CO—(C₆-C₂₄-aryl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring.

In one embodiment of the invention, ARYL preferably represents a phenyl radical which may be optionally substituted by one to three identical or different residues selected from the group: C₁-C₁₀-alkyl, C₂-C₁₀-alkenyl, C₁-C₁₀-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₁-C₁₀-alkyl), —OCOO—-(C₇-C₁₅-arylalkyl), —OCOO—(C₆-C₂₄-aryl), —SO₂—(C₇-C₁₅-arylalkyl), —SO₃—(C₇-C₁₅-aryl alkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₁-C₁₀-alkyl), —CO—(C₆-C₂₄-aryl), —SO₂—NH—(C₇-C₁₅-arylalkyl), —SO₂N14-(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring.

In another embodiment of the invention, ARYL preferably represents a pyridyl, pyrimidyl, pyridazinyl or pyrazinyl radical which may optionally be substituted by one to three identical or different radicals selected from the group: C₁-C₁₃-alkyl, C₂-C₁₀-alkenyl, C₁-C₁₀-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₆-C₂₄-aryl), —SO₂—NH—(C₁-C₁₀-alkyl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring.

In another embodiment of the invention, ARYL more preferably represents a pyridyl radical which may optionally be substituted by one to three identical or different radicals selected from the group C₁-C₁₀-alkyl, C₂-C₁₀-alkenyl, C₁-C₁₀-alkoxy, C₁-C₁₀-perhaloalkyl, C₂-C₁₀-alkynyl, C₆-C₂₄-aryl, C₃-C₁₅-hetaryl, —COO—(C₁-C₁₀-alkyl), —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₆-C₂₄-aryl), —SO₂—NH—(C₁-C₁₀-alkyl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring.

In a particularly preferred embodiment of the invention, ARYL represents 2-, 3- or 4-pyridinyl.

R¹ or/and R⁷ each preferably represent —COOR³ or —SO₂—R⁴, where R³ and R⁴ may be the same or different and are each selected from the group: C₁-C₁₀-alkyl, C₁-C₁₀-alkenyl, C₁-C₁₀-perhaloalkyl, C₇-C₁₅-arylalkyl or C₆-C₂₄-aryl, or R³ or R⁴ is C₁-C₁₀-alkyl or C₆-C₂₄-aryl each singly or multiply but not wholly substituted by C₁-C₁₀-alkoxy, Cl, Br or F. Preferably, R³ represents C₁-C₁₀-alkyl, C₆-C₂₄-aryl or C₂-C₁₀-alkenyl. More preferably, R³ represents tert-butyl. R⁴ preferably represents a C₁-C₁₀-alkyl or C₆-C₂₄-aryl each singly or multiply but not fully substituted by C₁-C₁₀-alkoxy, Cl, Br or F, or R⁴ represents C₁-C₁₀-perhaloalkyl. It is particularly preferable for R¹ or/and R⁷ to each represent —(CO)—O-(tert-butyl), —(CO)—O-(allyl), —(CO)—O-(methyl), —(CO)—O-(ethyl), —(CO)—O-(s-propyl), —(CO)—O-(n-propyl), —(CO)—O-(n-butyl), —(CO)—O-(s-butyl), butyl), —(CO)—O-(neopentyl), —(CO)—O-(nonafluorobutyl), —SO₂-(benzyl), —SO₂-(dimethylbenzyl), —SO₂-(trimethylbenzyl), —SO₂-(phenyl), —SO₂-(o-tolyl), —SO₂-(p-tolyl), —SO₂-(m-tolyl), —SO₂-(difluorobenzyl) or —SO₂-(trifluorobenzyl). It is very particularly preferable for R¹ or/and R⁷ to each represent —COO-(tert-butyl).

Preferably, R¹ and R⁷ are the same.

Preferably, R² represents C₁-C₁₀-alkyl, C₂-C₁₀-alkenyl, C₃-C₁₆-hetaryl or C₆-C₂₄-aryl, which may each be optionally further substituted by radicals selected from the group: C₁-C₁₀-alkyl, C₁-C₁₀-alkenyl, C₁-C₁₀-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₁-C₁₀-alkyl), —CO—(C₆-C₂₄-aryl), —SO₂—NH—(C₁-C₁₀-alkyl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰) together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring, —NCO or —NCS. More preferably, R² represents C₁-C₁₀-alkyl, C₂-C₁₀-alkenyl, C₃-C₁₆-hetaryl, C₇-C₁₅-arylalkyl or C₆-C₂₄-aryl. It is very particularly preferable for R² to represent methyl, ethyl, s-, n-propyl, n-, s-, tert-butyl, neopentyl, cyclohexyl, benzyl, o-, m-, p-tolyl or phenyl. It is even more preferable for R² to represent s-propyl.

Me preferably constitutes Mg.

Y preferably constitutes C₁-C₁₀-alkyl, F, Cl, Br or I. It is particularly preferable for Y to represent Cl or Br.

X preferably constitutes Br, Cl, I or —OSO₂—R⁸ where R⁸ is C₁-C₁₀-alkyl, C₁-C₁₀-perhaloalkyl, C₇-C₁₅-arylalkyl or C₆-C₁₄-aryl or R⁸ is C₁-C₁₀-alkyl or C₆-C₂₄-aryl each singly or multiply but not wholly substituted by C₁-C₁₀-alkoxy, Cl, Br or F. It is particularly preferable for X to represent Cl or Br.

Elemental iron or an iron compound constitutes an iron source within the meaning of the invention. The iron source within the meaning of the invention serves as catalyst. The iron source used can be any iron compounds of oxidation states −2, −1, 0, +1, +2, +3 or elemental iron. Useful iron compounds include, for example, iron complex compounds such as, for example, ferrocene, iron(II) phthalocyanine or iron pentacarbonyl or inorganic iron compounds such as, for example, iron(II) halides, for example iron(II) fluoride, iron(II) chloride or iron(II) bromide or, for example, iron(III) halides such as, for example, iron(III) fluoride, iron(III) chloride or iron(III) bromide or hydrated iron(II) or iron(III) halides such as, for example, iron(III) chloride hexahydrate or iron(II) chloride tetrahydrate or iron(II) or iron(III) nitrates, sulphates, phosphates, carbonates, perchlorates or organic iron compounds such as, for example, iron(II) or iron(III) acetate, formate, oxalate, acetylacetonates, stearate, pivalate or gluconate, or mixtures thereof. Preference for use as iron compound is given to elemental iron or inorganic iron compounds such as preferably iron(II) or iron(III) nitrates, sulphates, phosphates, carbonates, perchlorates or organic iron compounds such as iron(II) or iron(III) acetate, formate, oxalate, acetylacetonates, stearate, pivalate or gluconate, or mixtures thereof. Particular preference for use as iron sources is given to iron(II) or iron(III) halides or iron(III) or iron(II) acetylacetonates, most preferably iron(III) acetylacetonate.

Alkyl, alkenyl, alkoxy and alkynyl each independently represent a straight-chain, cyclic or branched alkyl, alkenyl, alkoxy or alkynyl radical, respectively. The same holds for the non-aromatic part of an arylalkyl radical.

C₁-C₁₀-Alkyl represents for example methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and 1-ethyl-2-methylpropyl. Preferably, C₁-C₁₀-alkyl represents methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl and n-hexyl.

Illustratively and preferably, C₂-C₁₀-alkenyl represents vinyl, allyl, isopropenyl, cyclohexenyl, cyclopentenyl and n-but-2-en-1-yl.

C₁-C₁₀-Alkoxy illustratively and preferably represents methoxy, ethoxy, n-propoxy, isopropoxy, n-butoxy, sec-butoxy and tert-butoxy, n-pentoxy, 1-methylbutoxy, 2-methylbutoxy, 3-methylbutoxy, neopentoxy, 1-ethylpropoxy, cyclohexoxy, cyclopentoxy and n-hexoxy.

Illustratively and preferably C₂-C₁₀-alkynyl represents ethynyl, propynyl, 1-butynyl, 2-butynyl or 3-butynyl.

C₆-C₂₄-Aryl herein represents a mono-, bi- or tricyclic carbocyclic aromatic radical having preferably 6 to 24 aromatic carbon atoms. Furthermore, the carbocyclic aromatic radicals can be substituted with up to five identical or different substituents per cycle, selected from the group C₁-C₁₀-alkyl, C₂-C₁₀-alkenyl, C₁-C₆-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₉-alkyl), —COO—(C₆-C₂₄-aryl), —SO₂(C₁-C₁₀-alkyl), —SO₂(C₆-C₂₄-aryl), —CO—(C₁-C₁₀-alkyl), —CO—(C₆-C₂₄-aryl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring. Illustratively and preferably C₅-C₂₄-aryl represents biphenyl, phenyl, o-, p-tolyl, naphthyl, phenanthrenyl, anthracenyl, acenaphthylene and fluorenyl.

C₇-C₁₅-Arylalkyl denotes in each case independently a straight-chain, cyclic or branched alkyl radical which conforms to the above definition and can be singly, multiply or fully substituted by aryl radicals according to the above definition. Illustratively and preferably C₇-C₁₅-arylalkyl represents benzyl, 1-phenylethylene, 1-phenylpropylene, 2-phenylpropylene, 1-phenylbutylene or 3-phenyl-2-methylpropylene.

A 3- to 7-membered saturated or partially unsaturated heterocycle herein represents a heterocycle which has up to 3 identical or different heteroatoms from the series S, N and/or O and which is linked via a ring carbon atom or a ring nitrogen atom and which may contain one or two double bonds. Preference is given to a 5- to 7-membered saturated heterocycle having up to 2 identical or different heteroatoms from the series S, N and/or O, Suitable examples include tetrahydrofur-2-yl, tetrahydrofur-3-yl, pyrrolidin-2-yl, pyrrolidin-3-yl, piperidin-1-yl, piperidin-4-yl, 1,2-dihydropyridin-1-yl, 1,4-dihydropyridin-1-yl, piperazin-1-yl, morpholin-4-yl, thiomorpholin-4-yl, azepin-1-yl, 1,4-diazepin-1-yl. Preference is given to piperidinyl, piperazinyl, morpholinyl and pyrrolidinyl.

C₁-C₁₀-Perhaloalkyl represents a C₁-C₁₀-alkyl radical fully substituted by halogen atoms. C₁-C₁₀-perhaloalkyl preferably represents a C₁-C₁₀-perfluoroalkyl. Illustratively and preferably C₁-C₁₀-perhaloalkyl represents trifluoromethyl, trichloromethyl, tribromomethyl, pentafluoroethyl, heptafluoropropyl, cyclononafluoropentyl, cyclononachlorocyclopentyl, heptafluoroisopropyl and nonafluorobutyl. Preferably C₁-C₁₀-perfluoroalkyl represents difluoromethyl, trifluoromethyl, trichloromethyl, tribromomethyl, pentafluoroethyl, heptafluoroisopropyl and nonafluorobutyl. It is very particularly preferable for C₁-C₁₀-perhaloalkyl or/and C₁-C₁₀-perfluoroalkyl to represent trifluoromethyl, pentafluoroethyl or heptafluoroisopropyl.

C₃-C₁₆-Hetaryl herein represents an aromatic heterocycle having up to 3 identical or different heteroatoms from the series S, N and/or O, which is linked via a ring carbon atom of the heteroaromatic, optionally also via a ring nitrogen atom of the heteroaromatic, and which has between 3 and 16 carbon atoms (C₃-C₁₆-hetaryl), preferably 3 to 7 (C₃-C₇) carbon atoms and more preferably 4 to 5 (C₄-C₅) carbon atoms (C₄-C₅-hetaryl). C₃-C₁₆-Hetaryl, C₃-C₇-hetaryl and C₄-C₅-hetaryl always have an at least sufficient number of heteroatoms for the heteroaromatic to be aromatic. A C₃-hetaryl thus has three carbon atoms and at least two nitrogen atoms or one nitrogen atom and one oxygen atom, or one nitrogen atom and one sulphur atom. C₃-C₁₆-Hetaryl may further be substituted by radicals selected from the group C₁-C₁₀-alkyl, C₂-C₁₀-alkenyl, C₁-C₁₀-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₆-C₂₄-aryl), —SO₂—NH—(C₁-C₁₀-alkyl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring. Illustratively and preferably there may be mentioned as C₃-C₁₆-hetaryl: pyridyl, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl, benzofuranyl or dibenzofuranyl.

C₁-C₈-Mono- or -dialkylamino herein represents an amino group substituted with one or two identical or different, cyclic, straight-chain or branched alkyl substituents, which each preferably have to 8 carbon atoms.

Illustratively and preferably C₁-C₈-monoalkylamino represents methylamino, ethylamino, n-propylamino, isopropylamino, t-butylamino, n-pentylamino and n-hexylamino.

Illustratively and preferably C₁-C₈-dialkylamino represents N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

The purview of the invention encompasses all the hereinabove and hereinbelow recited general or preferred definitions of radicals, parameters and elucidations among themselves, i.e. including between the respective ranges and preferences in any desired combination.

The present invention process for preparing the compounds of formula (1) can be carried out in the presence or absence of a solvent. The present invention process for preparing the compounds of formula (1) is preferably carried out in the presence of an organic solvent. The present invention process for preparing the compounds of formula (1) can be carried out in any organic, inert solvent. Illustratively and preferably there can be used as organic solvents linear, cyclic and branched aliphatic hydrocarbons, for example, pentane, hexane, heptane, octane, isooctane or cyclohexane or aromatic hydrocarbons, for example benzene, toluene, xylene, ethylbenzene, or mesitylene or ethers such as for example 1,4-dioxane, tetrahydrofuran, methyltetrahydrofuran, dibutyl ether, methyl t-butyl ether, diisopropyl ether, diethylene glycol dimethyl ether, dimethoxymethane or amines, such as tetramethylurea, N,N,N′,N′-tetramethylethylenediamine or amides such as for example dimethylformamide, diethylformamide, N-methylpyrrolidone, dimethylacetamide or dimethyl sulphoxide or sulpholane or organic carbonates such as for example propylene carbonate or diethyl carbonate or mixtures thereof. Particular preference is given to using ethers, such as, more particularly, dioxane, tetrahydrofuran, tert-butyl methyl ether, amines, such as, more particularly, N,N,N′,N′-tetramethylethylenediamine or amides such as, more particularly, N-methylpyrrolidone, dimethylformamide, diethylformamide, dimethylacetamide or dimethyl sulphoxide or sulpholane or mixtures thereof. Very particular preference for use as organic solvent is given to N-methylpyrrolidone or an organic solvent mixture that contains N-methylpyrrolidone.

The temperatures at which the present invention process for preparing the compounds of formula (1) is carried out are for example between −100° C. and 50° C., preferably between −50° C. and 10° C. and more preferably between −20° C. and +10′C.

The process of the present invention is generally carried out at standard pressure. In general, the process can be carried out at any desired pressure.

In the process of the present invention, the iron sources are used in amount of substance ratios based on the compounds of formula (3) ranging from 50:1 to 1:50, preferably from 30:1 to 1:30 and more preferably from 20:1 to 1:20.

In the present invention process for preparing the compounds of formula (1), the amount of substance ratios of the compounds of formula (2) and of the compounds of formula (3) are between 1:5 and 5:1, preferably between 1:5 and 1:1 and more preferably between 1:3 and 1:1.

The present invention process for preparing the compounds of formula (1) from the compounds of formula (2) is carried out under substantially anhydrous conditions. Substantially anhydrous means that the water content is preferably between 0.0001% by weight and 0.1% by weight, based on the amount of reaction mixture used.

The present invention process for preparing the compounds of formula (1) is preferably carried out by initially charging the compounds of formula (2), the iron source and the solvent. Thereafter, the reaction mixture is generally inertised, for example by displacing the air with anhydrous nitrogen or argon. Then, the compounds of formula (3) for example are added, preferably in metered fashion. The end of the reaction can be determined using analytical methods known to a person skilled in the art, for example chromatography. The rest of the working-up is done by employing common methods known to a person skilled in the art for hydrolysing the products from Grignard reactions, by adding catalytic amounts of water or water-containing compounds, for example saturated salt solutions and optionally further purification via extraction with organic solvents or/and crystallisation for example. The above addition of the starting materials can likewise be carried out in some other order, or concurrently. Preferably, the compound of formula (1) is further purified by recrystallisation with organic solvents.

The compounds of formula (1) can also form in the form of their ammonium salts. The ammonium salts of compounds of formula (1) can be converted into the free compounds of formula (1) using common methods known to a person skilled in the art, for example and preferably via reaction or titration with carboxylic acids, more particularly citric acid.

The invention further comprises a process for preparing the compounds of formula (4),

NH₂-ARYL-R² (4)

where ARYL and R² are each as defined above, wherein the compounds of formula (1) are reacted in the presence of at least one acid or at least one base to form compounds of formula (4) or salts thereof.

Bases within the meaning of the invention for preparing the compounds of formula (4) from the compounds of formula (1) in the process of the present invention are for example alkaline earth or alkali metal carbonates, hydroxides, hydrogenphosphates, phosphates or tertiary amines.

Acids within the meaning of the invention for preparing the compounds of formula (4) from the compounds of formula (1) are, for example, sulphuric acid, nitric acid, hydrohalic acids such as hydrochloric acid or hydrobromic acid, phosphoric acids such as orthophosphoric acid, sulphamic acid, also organic acids, more particularly aliphatic, alicyclic, aromatic or heterocyclic mono- or polybasic carboxylic, sulphonic or sulphuric acids, e.g. formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methanesulphonic acid, ethanesulphonic acid, ethanedisulphonic acid, 2-hydroxyethanesulphonic acid, benzenesulphonic acid, p-toluenesulphonic acid, naphthalenemonosulphonic acid, naphthalenedisulphonic acid or laurylsulphuric acid. Hydrohalic acids are preferably used as acids, e.g. HCl or HBr. It is very particularly preferred to use HCl as an acid in the process of the present invention.

In general, the compounds of formula (4) can be prepared from the compounds of formula (1) using common methods known to a person skilled in the art for deprotecting amines out of amides or carbamides.

The compounds of formula (4) can likewise be present in the form of their ammonium salts. Therefore, the invention likewise comprises a process for preparing the compounds of formula (4) wherein the compounds of formula (4) are present in the form of their ammonium salts.

The compounds of formula (2) are obtainable using methods known to a person skilled in the art, as described in Journal of Organic Chemistry 2008, 73, 6025-6028 for example.

The compounds of formula (4) are obtainable from an aminohaloaryl compound, more particularly from amino-2-chloropyridine using the present invention process for preparing the compounds of formula (1) in a process proceeding from compounds of formula (5)

NH₂-ARYL-X (5)

where ARYL is as defined above and X is Cl, Br, I or —OSO₂—R⁸ where R⁸ is C₁-C₁₀-alkyl, C₁-C₁₀-perhaloalkyl, C₇-C₁₅-arylalkyl or C₆-C₂₄-aryl or R⁸ is C₁-C₁₀-alkyl or C₆-C₂₄-aryl each singly or multiply but not wholly substituted by C₁-C₁₀-alkoxy, Cl, Br or F.

The invention therefore likewise comprises a process for preparing the compounds of formula (4) or salts thereof,

NH₂-ARYL-R² (4)

where ARYL represents a substituted or unsubstituted carbocyclic C₆-C₂₄-aryl radical or a substituted or unsubstituted heteroaromatic C₃-C₁₆-hetaryl radical, and
R² is C₁-C₁₀-alkyl, C₂-C₁₀-alkenyl, C₆-C₂₄-aryl, C₇-C₁₅-arylalkyl, C₃-C₁₆-hetaryl or a 3- to 7-membered saturated or partially unsaturated heterocycle which may optionally be further substituted by radicals selected from the group: C₁-C₁₀-alkyl, C₂-C₁₀-alkenyl, C₁-C₁₀-alkoxy, C₁-C₁₀-perhaloalkyl, C₂-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₄-C₂₄-aryl), —SO₂—NH—(C₁-C₁₀-alkyl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring, in which in a step a) compounds of formula (5)

NH₂-ARYL-X (5)

where ARYL is as defined above and X is Cl, Br, I or —OSO₂—R⁸ where R⁸ is C₁-C₁₀-alkyl, C₁-C₁₀-perhaloalkyl, C₇-C₁₅-arylalkyl or C₆-C₂₄-aryl or R⁸ is C₁-C₁₀-alkyl or C₆-C₂₄-aryl each singly or multiply but not wholly substituted by C₁-C₁₀-alkoxy, Cl, Br or F, are reacted with compounds of formula (6)

R¹—Z (6)

where R¹ is —COOR^{S} or —SO₂—R⁴, where R³ and R⁴ may be the same or different and are each selected from the group: C₁-C₁₀-alkyl, C₂-C₁₀-alkenyl, C₁-C₁₀-perhaloalkyl, C₇-C₁₅-arylalkyl or C₆-C₂₄-aryl, or R³ or R⁴ is C₁-C₁₀-alkyl or C₆-C₂₄-aryl each singly or multiply but not wholly substituted by C₁-C₁₀alkoxy, Cl, Br or F, or R¹ is —SO₂—NH—(C₁-C₁₀-alkyl), —SO₂—NH—(C₇-C₁₅-arylalkyl), —SO₂NH—(C₆-C₂₄-aryl) or —SO₂(NR⁵R⁶), where R⁵ and R⁶ each represent C₁-C₁₀-alkyl, or NR⁵R⁶ together form a 5- to 7-membered ring, and

Z represents fluorine, chlorine, bromine, iodine or optionally substituted or unsubstituted —O—CO₂—(C₁-C₁₀-alkyl), —O—CO₂(C₆-C₂₄-aryl), —O—CO₂—(C₇-C₁₅-aryl alkyl), —OSO₂(C₁-C₁₀-alkyl), —OSO₂(C₆-C₂₄-aryl), —OSO₂—NH—(C₇-C₁₅-arylalkyl) or —OSO₂—(C₇-C₁₅-arylalkyl) to form compounds of formula (7)

W-ARYL-X (7)

where ARYL and X are each as defined above and W is —NHR¹ or —N(R¹)₇, where R¹ is as defined above, and in a step b) the compounds of formula (7) are reacted with compounds of formula (3)

R²-MeY (3)

where R² is as defined above and Y is an anionic ligand and Me is a metal selected from the group Mg, Cu Mn, Zn, in the presence of at least one iron source to form compounds of formula (1), and in a step c) the compounds of formula (1) are reacted in the presence of acids or bases to form compounds of formula (4).

Z is preferably fluorine, chlorine, bromine, iodine, —O—CO₂—(C₁-C₁₀-alkyl), —O—CO₂(C₆-C₂₄-aryl), —O—CO₂—(C₇-C₁₅-arylalkyl), —OSO₂(C₁-C₁₀-alkyl), —OSO₂(C₆-C₂₄-aryl). Z is more preferably —O—CO₂—(C₁-C₁₀-alkyl) or —O—CO₂(C₆-C₂₄-aryl) and R¹ is then —COOR³ where R³ is C₁-C₁₀-alkyl or C₆-C₂₄-aryl each singly or multiply but not fully substituted by C₁-C₁₀-alkoxy, Cl, Br or F. It is very particularly preferable for Z to be —OCOO-(tert-butyl).

The compounds of formula (6) preferably constitute di-tert-butyl dicarbonate, allyl chloroformate, benzyl chloroformate, p-toluenesulphonyl chloride, o-toluenesulphonyl chloride, m-toluenesulphonyl chloride, methanesulphonyl chloride, trifluoromethanesulphonyl chloride or ethanesulphonyl chloride. It is particularly preferable for the compound of formula (6) to constitute di-tert-butyl dicarbonate.

The invention preferably comprises a process for preparing the compounds of formula (8)

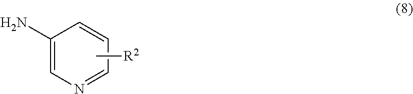

where R² is C₁-C₁₀-alkyl, C₂-C₁₀-alkenyl, C₆-C₂₄-aryl, C₇-C₁₅-arylalkyl or C₃-C₁₆-hetaryl and wherein in a step a) compounds of formula (9)

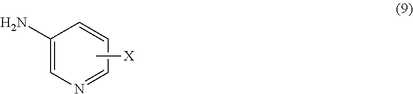

where X is Cl, Br, I or —OSO₂—R⁸ where R⁸ is C₁-C₁₀-alkyl, C₁-C₁₀-perhaloalkyl, C₇-C₁₅-arylalkyl or C₆-C₂₄-aryl or R⁸ is C₁-C₁₀-alkyl or C₆-C₂₄-aryl each singly or multiply but not wholly substituted by C₁-C₁₀-alkoxy, Cl, Br or F, are reacted with compounds of formula (6)

R¹—Z (6)

where R¹ is —COOR³ or —SO₂—R⁴, where R³ and R⁴ may be the same or different and are each selected from the group: C₁-C₁₀-alkyl, C₂-C₁₀-alkenyl, C₁-C₁₀-perhaloalkyl, C₇-C₁₅-arylalkyl or C₆-C₂₄-aryl, or R³ or R⁴ is C₁-C₁₀-alkyl or C₆-C₂₄-aryl each singly or multiply but not wholly substituted by C₁-C₁₀-alkoxy, Cl, Br or F, or R¹ is —SO₂—NH—(C₁-C₁₀-alkyl), —SO₂—NH—(C₇-C₁₅-arylalkyl), —SO₂NH—(C₆-C₂₄-aryl) or —SO₂(NR⁵R⁶), where R⁵ and R⁶ each represent C₁-C₁₀-alkyl, or NR⁵R⁶ together form a 5- to 7-membered ring, and

Z represents fluorine, chlorine, bromine, iodine or optionally substituted or unsubstituted —O—CO₂—(C₁-C₁₀-alkyl), —O—CO₂(C₆-C₂₄-aryl), —O—CO₂—(C₇-C₁₅-arylalkyl), —OSO₂(C₁-C₁₀-alkyl), —OSO₂(C₆-C₂₄-aryl), —OSO₂—NH—(C₇-C₁₅-arylalkyl) or —OSO₂—(C₇-C₁₅-arylalkyl) to form compounds of formula (10)

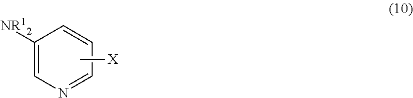

where R¹ and X are each as defined above, and the compounds of formula (10) are reacted in a step b) with compounds of formula (3)

R²-MeY (3)

where R² is as defined above and Y is an anionic ligand and Me is a metal selected from the group Mg, Ca, Mn, Zn in the presence of at least one iron source to form compounds of formula (11)

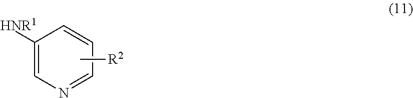

where R¹ and R² are each as defined above, and the compounds of formula (11) are reacted in a step c) in the presence of acids or bases to form compounds of formula (8) or salts thereof.

The invention more preferably comprises a process for preparing the compounds of formula (12)

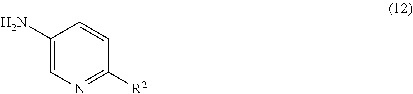

where R² is C₁-C₁₀-alkyl, C₁-C₁₀-alkenyl, C₆-C₂₄-aryl, C₇-C₁₅-arylalkyl or C₃-C₁₆-hetaryl and wherein in a step a) compounds of formula (13)

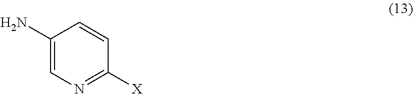

where X is Cl, Br, I or —OSO₂—R⁸ where R⁸ is C₁-C₁₀-alkyl, C₁-C₁₀-perhaloalkyl, C₇-C₁₅-arylalkyl or C₆-C₂₄-aryl or R⁸ is C₁-C₁₀-alkyl or C₆-C₂₄-aryl each singly or multiply but not wholly substituted by C₁-C₁₀-alkoxy, Cl, Br or F, are reacted with compounds of formula (14)

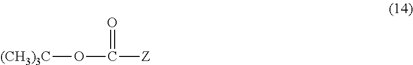

where Z represents fluorine, chlorine, bromine, iodine or optionally substituted or unsubstituted —O—CO₂—(C₁-C₁₀-alkyl), —O—CO₂(C₆-C₂₄-aryl), —O—CO₂—(C₇-C₁₅-arylalkyl), —OSO₂(C₁-C₁₀-alkyl), —OSO₂(C₆-C₂₄-aryl), —OSO₂—NH—(C₇-C_{:5}-arylalkyl) or —OSO₂—(C₇-C₁₅-arylalkyl) to form compounds of formula (15)

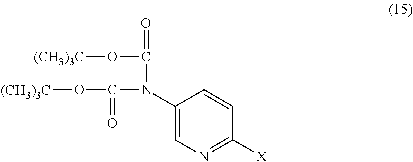

where X is as defined above, and the compounds of formula (15) are reacted in a step b) with compounds of formula (3)

R²-MeY (3)

where R² is as defined above and Y is an anionic ligand and Me is a metal selected from the group Mg, Ca, Mn, Zn in the presence of at least one iron source to form compounds of formula (16)

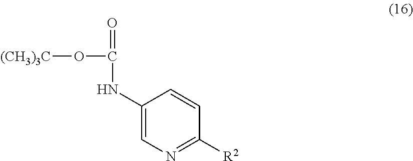

and the compounds of formula (16) are reacted in a step c) in the presence of acids or bases to form compounds of formula (12) or salts thereof.

It is very particularly preferable for the compounds of formula (12) and the compounds of formula (8) and the compounds of formula (4) to be 5-amino-2-isopropylpyridine. It is very particularly preferable for the compounds of formula (13) and the compounds of formula (9) and the compounds of formula (5) to be 5-amino-2-chloropyridine.

The compounds of formula (14) are preferably di-(tert-butyl) dicarbonate.

Since the preparation of the compounds of formulae (8) and (12) are each preferred embodiments of the preparation of the compounds of formula (4), the hereinbelow described present invention processes as per steps a), b) and c) hold for the preparation of the compounds of formulae (4), (8) and (12).

The present invention process as per step a) can be carried out in the presence of bases or in the absence of bases. The process is preferably carried out in the absence of additional bases.

Useful bases for the present invention process as per step a) include for example hydrogencarbonates, such as sodium hydrogencarbonate and potassium hydrogencarbonate, alkali metal hydroxides or alkoxides, for example sodium methoxide, potassium tert-butoxide, sodium hydroxide, potassium hydroxide or organic bases, for example pyridine, ammonium compounds, for example ammonium hydroxide or and tertiary amines, such as trimethylamine, triethylamine, tributylamine, N,N-dimethylaniline, N,N-dimethylbenzylamine, pyridine, N-methylpyridine, N-methylmorpholine, N,N-dimethylaminopyridine, diazabicyclooctane (DABCO), diazabicyclononene (DBN) and diazabicycloundecene (DBU) or mixtures thereof.

The present invention process as per step a) is preferably carried out in the presence of an organic solvent. Suitable organic solvents for carrying out the process of the present invention are more particularly apolar alicyclic or aromatic hydrocarbons such as, for example, benzene, toluene, xylene, n-pentane, isopentane, hexane, heptane, octane, isooctane, cyclohexane, cyclopentane, cycloheptane, cyclononane, cyclooctane, methylcyclopentane, methylcyclohexane, bicyclo[4.1.0]beptane or mixtures thereof. Particularly preferred solvents are organic, apolar branched or unbranched, optionally cyclic, aliphatic hydrocarbons, more particularly hexane, heptane, octane, cyclohexane, methylcyclohexane or isooctane. It is very particularly preferable to use n-heptane as solvent.

Step a) of the process of the present invention is carried out at temperatures between 20° C. and 200° C. Preferably, step a) of the process of the present invention is carried out at temperatures between 50° C. and 130° C.

The amount of substance ratio of the compounds of formula (6) to the compounds of formula (5) is between 5:1 and 1:5, preferably between 3:1 and 1:3 and more preferably between 3:1 and 1:1.

Step a) of the process of the present invention is preferably carried out by initially charging the compounds of formula (5), optionally in the presence of the organic solvent, and then adding the compounds of formula (6), preferably dissolved in the organic solvent, in metered fashion. Thereafter, the mixture is heated. Preferably, portions of the solvent are distillatively removed from the reaction mixture during the reaction. Preferably, in this case, the reaction mixture is replenished with a corresponding amount of the solvent, optionally and preferably containing compounds of formula (6). The compounds of formula (7) can be further purified via crystallisation for example.

The process procedure, temperatures and preferences indicated for preparing the compounds of formula (1) likewise hold for step b) of the process of the present invention.

Step c) of the process of the present invention can be carried out in the presence or absence of organic solvents. Preferably, step c) of the process of the present invention is carried out in the presence of an organic solvent.

The organic solvents used in step c) of the process of the present invention are preferably linear, cyclic or branched aliphatic hydrocarbons, for example pentane, hexane, heptane, octane, iso-octane or cyclohexane or aromatic hydrocarbons, for example benzene, toluene, xylene, ethylbenzene, mesitylene or, for example, ketones, alcohols, for example isopropanol, ethanol, n-, s-, i-butanol or sulphones or amides. It is particularly preferable to use alcohols, more particularly isopropanol, as solvent in step c) of the process of the present invention.

Bases within the meaning of step c) of the process of the present invention are for example alkaline earth or alkali metal carbonates, hydroxides, hydrogenphosphates, phosphates or tertiary amines.

Acids within the meaning of step c) of the process of the present invention are for example sulphuric acid, nitric acid, hydrohalic acids such as hydrochloric acid or hydrobromic acid, phosphoric acids such as orthophosphoric acid, sulphamic acid, also organic acids, more particularly aliphatic, alicyclic, aromatic or heterocyclic mono- or polybasic carboxylic, sulphonic or sulphuric acids, e.g. formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methanesulphonic acid, ethanesulphonic acid, ethanedisulphonic acid, 2-hydroxyethanesulphonic acid, benzenesulphonic acid, p-toluenesulphonic acid, naphthalenemonosulphonic acid, naphthalenedisulphonic acid or laurylsulphuric acid. Hydrohalic acids are preferably used as acids, e.g. HCl or HBr. It is very particularly preferred to use HCl as an acid in the process of the present invention.

Temperatures at which step c) of the process of the present invention is carried out are preferably between 10° C. and 100° C. and more preferably between 20° C. and 70° C.

Step c) of the process of the present invention is preferably carried out by the compounds of formula (1) being initially charged and optionally mixed with the solvent. Thereafter, the acids or bases are added, preferably in metered fashion, as a solution or without a solvent. The compounds of formula (4) can be further purified by distillation, crystallisation or extraction for example. Preferably, the compounds of formula (4), which are generally in the form of their salts after the reaction of compounds of formula (1), are converted back into the salt-free compounds of formula (4) by reaction with acids or bases. The further purification of the salt-free compounds of formula (4) is preferably accomplished via distillation. The further purification of the salt-free compounds of formula (1) is preferably accomplished via distillation.

The end of the reactions as per steps a), b) and c) of the process of the present invention can be determined using common methods known to a person skilled in the art.

The feedstocks and reactants used in the processes of the present invention are either obtainable using methods known to a person skilled in the art, or commercially available.

The process of the present invention provides the compounds of formula (1) in good yields, efficiently on an industrial scale. Moreover, the compounds of formula (4), which are significant intermediates in the manufacture of medicinal products, are likewise obtainable in an efficient manner from the compounds of formula (1) by protective group elimination with acids or bases.

The examples which follow illustrate the invention and shall not be construed as limiting it.

### EXAMPLES

### 1. Preparation of 5-(di-Boc-amino)-2-ehloropyridine

100 g (0.76 mol) of 5-amino-2-chloropyridine are admixed with 120 mL (0.82 mol) of n-heptane and also 200 g (0.89 mol) of di-tert-butyl dicarbonate at room temperature. The resulting suspension is heated up to 60° C. over 2 h and stirred at 60° C. for 5 h. Then, a solution of 50 g (0.22 mol) of di-tert-butyl dicarbonate in 30 mL (0.20 mol) of n-heptane is added, which is followed by heating to reflux and removing distillate until the internal temperature reaches 115° C. This is followed by stirring at 115° C. for 1 h with distillate removal. Addition of 240 mL (1.64 mol) of n-heptane is followed by heating to reflux and removing distillate until the internal temperature reaches 100° C. This is followed by uniform metered addition, under reflux, of a solution of 200 g (0.89 mol) of di-tert-butyl dicarbonate in 120 mL (0.82 mol) of n-heptane over about 12 h, and stirring under reflux for a further 4 h.

The batch is slowly cooled down to room temperature and stirred at room temperature for 1 h. The precipitated product is filtered off and the filter cake is washed twice with 120 mL (0.82 mol) of n-heptane each time. The moist product obtained is dried in a vacuum drying cabinet at about 60° C. and <100 mbar to constant weight.

Yield: 241.4 g (0.73 mol, 96%)

### 2. Preparation of 5-(Boc-amino)-2-isopropylpyridine

At room temperature, 50 g (0.15 mol) of 5-(di-Boc-amino)-2-chloropyridine, 140 g (1.94 mol) of tetrahydrofuran and 10 g (0.10 mol) of N-methylpyrrolidone are initially charged under nitrogen atmosphere. The resulting solution is precooled down to about −20° C. Subsequently, a solution of 3.0 g (8.2 mmol) of iron(III) acetylacetonate in 30 g (0.42 mol) of tetrahydrofuran is metered at −20° C. over 3 h concurrently with 215 g (0.42 mol) of isopropylmagnesium chloride, approximately 20% solution in tetrahydrofuran. On completion of the metered addition the batch is stirred at −20° C. for 15 min, then heated up to 0° C. during 1 h and stirred at 0° C. for 30 min. The batch is subsequently discharged onto 175 g (0.13 mol) of a 14.3% solution of citric acid at max. 10° C. and the reaction mixture is stirred at room temperature for 30 min. Following phase separation, the aqueous phase is extracted once with 40 g (0.13 mol) of xylene. The combined organic phases are washed once with 100 mL of a 5% solution of sodium hydrogencarbonate and then filtered through 40 g (bed height of about 3 cm) of silica gel. Subsequently, the filtrate is concentrated at 50° C. in vacuo down to about 100 mbar. The distillation bottoms are admixed with 60 g (0.61 mot) of methylcyclohexane at room temperature, and the resulting suspension is heated up to 85° C. The resulting clear reddish orange solution is cooled back down to room temperature and stirred at room temperature for 30 min. The precipitated product is filtered off and the filter cake is washed once with 30 g (0.31 mol) of methylcyclohexane. The moist product obtained is dried in a vacuum drying cabinet at about 60° C. and <100 mbar to constant weight.

Yield: 25.7 g (111 mol, 72%)

### 3. Preparation of 5-amino-2-isopropylpyridine

100 g (0.41 mol) of 5-(Boc-amino)-2-isopropylpyridine are slurried up at room temperature in 500 g (8.3 mol) of isopropanol. Then, 200 g (1.8 mol) of an approximately 33% solution of hydrogen chloride in isopropanol are metered at 25° C. On completion of the metered addition the batch is stirred at 25° C. for 30 min, then heated up to 50° C. over about 1 h and stirred at 50° C. for 3 h. Subsequently, 600 mL of distillate are removed under atmospheric pressure and 350 g (3.8 mol) of toluene are added to the resulting suspension at 40-50° C. The resulting suspension is discharged at room temperature onto a mixture of 150 g (8.3 mol) of completely ion-free water and 110 g (1.4 mol) of 50% aqueous sodium hydroxide solution and stirred at room temperature for 15 mM. Following phase separation, the organic phase is concentrated in vacuo and the remaining oil is fractionally distilled through a column at 8 mbar.

Yield: 47.7 g (0.35 mol, 85%)

What is claimed is:

1. Process for preparing the compounds of formula (1)
R¹—NH-ARYL-R² (1)
where R¹ is —COOR³ or —SO₂—R⁴, where R³ and R⁴ are each selected from the group: C₁-C₁₀-alkyl, C₂-C₁₀-alkenyl, C₁-C₁₀-perhaloalkyl, C₇-C₁₅-arylalkyl or C₆-C₂₄-aryl, or R³ or R⁴ is C₁-C₁₀-alkyl or C₆-C₂₄-aryl each singly or multiply but not wholly substituted by C₁-C₁₀-alkoxy, Cl, Br or F, or R¹ is —SO₂—NH—(C₁-C₁₀-alkyl), —SO₂—NH—(C₂-C₁₅-arylalkyl), —SO₂NH—(C₆-C₂₄-aryl) or —SO₂(NR⁵R⁶), where R⁵ and R⁶ each represent C₁-C₁₀-alkyl, or NR⁵R⁶ together form a 5- to 7-membered ring, and
ARYL represents a substituted or unsubstituted carbocyclic C₆-C₂₄-aryl radical or a substituted or unsubstituted heteroaromatic C₃-C₁₆-hetaryl radical, and
R² is C₁-C₁₀-alkyl, C₂-C₁₀-alkenyl, C₆-C₂₄-aryl, C₇-C₁₅-arylalkyl, C₃-C₁₆-hetaryl or a 3- to 7-membered saturated or partially unsaturated heterocycle which may optionally be further substituted by radicals selected from the group: C₁-C₁₀-alkyl, C₂-C₁₀-alkenyl, C₁-C₁₀-alkoxy, C₁-C₁₀-perhaloalkyl, C₁-C₁₀-alkynyl, C₆-C₂₄-aryl, C₃-C₁₆-hetaryl, —COO—(C₁-C₁₀-alkyl), —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₅-arylalkyl), —SO₃—(C₂-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₁-C₁₀-alkyl), —CO—(C₆-C₂₄-aryl), —SO₂—NH—(C₁-C₁₀-alkyl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent C₁-C₁₀-alkyl or C₆-C₂₄-aryl, or —NR¹¹R¹² together form a 5- to 7-membered ring,
characterized in that compounds of formula (2) 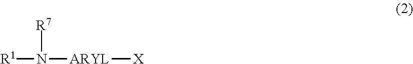
where R¹ and R⁷ may be the same or different and R¹ and ARYL are each as defined above and R⁷ is —COOR³ or —SO₂—R⁴, where R³ and R⁴ are each selected from the group: C₁-C₁₀-alkyl, C₂-C₁₀-alkenyl, C₁-C₁₀-perhaloalkyl, C₆-C₂₄-aryl or C₇-C₁₅-arylalkyl or R³ or R⁴ is C₁-C₁₀-alkyl or C₆-C₂₄-aryl each singly or multiply but not wholly substituted by C₁-C₁₀-alkoxy, Cl, Br or F, or R¹ is —SO₂—NH—(C₁-C₁₀-alkyl), —SO₂—NH—(C₇-C₁₅-arylalkyl), —SO₂NH—(C₆-C₂₄-aryl) or —SO₂(NR⁵R⁶), where R⁵ and R⁶ each represent C₁-C₁₀-alkyl, or NR⁵R⁶ together form a 5- to 7-membered ring, and
X is Cl, Br, I or —OSO₂—R⁸ where R⁸ is C₁-C₁₀-alkyl, C₁-C₁₀-perhaloalkyl, C₇-C₁₅-arylalkyl or C₆-C₂₄-aryl or R⁸ is C₁-C₁₀-alkyl or C₆-C₂₄-aryl each singly or multiply but not wholly substituted by C₁-C₁₀-alkoxy, Cl, Br or F
are reacted in the presence of at least one iron source
with compounds of formula (3)
R²-MeY (3)
where R² is as defined above and
Y is an anionic ligand and
Me is a metal selected from the group Mg, Ca, Mn, Zn,
to form compounds of formula (1) or salts thereof.

2. Process according to claim 1, characterized in that ARYL represents a five- or six-membered heteroaromatic ring which has a nitrogen atom and may be optionally substituted by 1, 2 or 3 substituents selected from the group C₁-C₁₀-alkyl, C₁-C₁₀-alkenyl, C₁-C₆-haloalkyl, C₁-C₆-alkylthio, C₆-C₂₄-aryl, C₃-C₁₆-hetaryl, 3- to 7-membered saturated or partially unsaturated heterocycle.

3. Process according to claim 1 or 2, characterized in that R¹ or/and R⁷ represents —(CO)—O-(tert-butyl), —(CO)—O-(allyl), —(CO)—O-(methyl), —(CO)—O-(ethyl), —(CO)—O-(s-propyl), —(CO)—O-(n-propyl), —(CO)—O-(n-butyl), —(CO)—O-(s-butyl), —(CO)—O-(i-butyl), —(CO)—O-(neopentyl), —(CO)—O-(nonafluorobutyl), —(CO)—O-(nonafluorobutyl), —SO₂-(benzyl), —SO₂—(dimethylbenzyl), —SO₂-(trimethylbenzyl), —SO₂-(phenyl), —SO₂-(o-tolyl), —SO₂-(p-tolyl), —SO₂-(m-tolyl), —SO₂-(difluorobenzyl) or —SO₂-(trifluorobenzyl).

4. Process according to one or more of claims 1 to 3, characterized in that R² represents C₁-C₁₀-alkyl, C₂-C₁₀-alkenyl, C₃-C₁₆-hetaryl, C₇-C₁ or C₆-C₂₄-aryl.

5. Process according to one or more of claims 1 to 4, characterized in that Me represents magnesium.

6. Process according to one or more of claims 1 to 5, characterized in that Y represents chloride, bromide or iodide.

7. Process according to one or more of claims 1 to 6, characterized in that X represents Cl, Br, I, —OSO₂(C₁-C₁₀-alkyl), —OSO₂(C₆-C₂₄-aryl) or —OSO₂—(C₇-C₁₅-arylalkyl).

8. Process according to one or more of claims 1 to 7, characterized in that organic iron compounds of oxidation state (III) are used as iron sources.

9. Process for preparing the compounds of formula (4)
NH₂-ARYL-R² (4)
where ARYL and R² are each as defined under claim 1, characterized in that the compounds of formula (1) are prepared according to one or more of claims 1 to 7 and are reacted in the presence of an acid or a base to form compounds of formula (4) or salts thereof.

10. Process according to claim 9, characterized in that hydrohalic acid, sulphonic acids or carboxylic acids are used as acids.

11. Process for preparing the compounds of formula (4) or salts thereof
NH₂-ARYL-R² (4)
where ARYL represents a substituted or unsubstituted carbocyclic C₆-C₂₄-aryl radical or a substituted or unsubstituted heteroaromatic C₃-C₁₆-hetaryl radical, and
R² is C₁-C₁₀-alkyl, C₂-C₁₀-alkenyl, C₆-C₂₄-aryl, C₇-C₁₅-arylalkyl, C₃-C₁₅-hetaryl or a 3- to 7-membered saturated or partially unsaturated heterocycle which may optionally be further substituted by radicals selected from the group: C₁-C₁₀-alkyl, C₂-C₁₀-alkenyl, C₁-C₁₀-alkoxy, C₁-C₁₀-perhaloalkyl, C₂-C₁₉-alkynyl, C₆-C₂₄-aryl, C₃-C₁₆-hetaryl, —COO—(C₇-C₁₅-arylalkyl), —OCOO—(C₁-C₁₀-alkyl), —OCOO—(C₇-C₁₅-arylalkyl), —OCOO—(C₆-C₂₄-aryl), —SO₂—(C₇-C₁₅-arylalkyl), —SO₃—(C₇-C₁₅-arylalkyl), —SO₃(C₆-C₂₄-aryl), —SO₃(C₁-C₁₀-alkyl), —COO—(C₆-C₂₄-aryl), —SO₂(C₁-C₁₀-alkyl), —SO₂(C₆-C₂₄-aryl), —CO—(C₆-C₂₄-aryl), —SO₂—NH—(C₇-C₁₅-arylalkyl), —SO₂NH—(C₆-C₂₄-aryl) or —SO₂(NR⁹R¹⁰), where R⁹ and R¹⁰ may be the same or different and each independently represent C₁-C₁₀-alkyl or C₆-C₂₄-aryl or NR⁹R¹⁰ together form a 5- to 7-membered ring, C₁-C₈-mono- or -dialkylamino, halogen, —OCO—(NR¹¹R¹²) or —CO—(NR¹¹R¹²), where R¹¹ and R¹² may be the same or different and each independently represent Q-C₁₀-alkyl or C₆-C₂₄-aryl, or —NR¹¹R¹² together form a 5- to 7-membered ring,
characterized in that in a step a) compounds of formula (5)
NH₂-ARYL-X (5)
where ARYL is as defined above and X is Cl, Br, I or —OSO₂—R⁸ where R⁸ is C₁-C₁₀-alkyl, C₁-C₁₀-perhaloalkyl, C₇-C₁₅-arylalkyl or C₆-C₂₄-aryl or R⁸ is C₁-C₁₀-alkyl or C₆-C₂₄-aryl each singly or multiply but not wholly substituted by C₁-C₁₀-alkoxy, Cl, Br or F,
are reacted with compounds of formula (6)
R¹—Z (6)
where R¹ is —COOR³ or —SO₂—R⁴, where R³ and R⁴ may be the same or different and are each selected from the group: C₁-C₁₀-alkyl, C₂-C₁₀-alkenyl, C₁-C₁₀-perhaloalkyl, C₇-C₁₅-arylalkyl or C₆-C₂₄-aryl, or R³ or R⁴ is C₁-C₁₀-alkyl or C₆-C₂₄-aryl each singly or multiply but not wholly substituted by C₁-C₁₀-alkoxy, Cl, Br or F, or R¹ is —SO₂—NH—(C₁-C₁₀-alkyl), —SO₂—NH—(C₇-C₁₅-arylalkyl), —SO₂NH—(C₆-C₂₄-aryl) or —SO₂(NR⁵R⁶), where R⁵ and R⁶ each represent C₁-C₁₀-alkyl, or NR⁵R⁶ together form a 5- to 7-membered ring, and
Z represents fluorine, chlorine, bromine, iodine or optionally substituted or unsubstituted —O—CO₂—(C₁-C₁₀-alkyl), —O—CO₂—(C₆-C₂₄-aryl), —O—CO₂—(C₇-C₁₅-arylalkyl), —OSO₂(C₁-C₁₀-alkyl), —OSO₂(C₆-C₂₄-aryl), —OSO₂—NH—(C₇-C₁₅-arylalkyl) or —OSO₂—(C₇-C₁₅-arylalkyl) to form compounds of formula (7)
W-ARYL-X (7)
where ARYL and X are each as defined above and W is —NHR¹ or —N(R¹)₂, where R¹ is as defined above, and
in a step b) the compounds of formula (7) are reacted with compounds of formula (3)
R²-MeY (3)
where R² is as defined above and Y is an anionic ligand and Me is a metal selected from the group Mg, Ca, Mn, Zn,
in the presence of at least one iron source to form compounds of formula (1), and
in a step c) the compounds of formula (1) are reacted in the presence of acids or bases to form compounds of formula (4).

12. Process according to one or more of claims 1 to 11, characterized in that the process according to step b) is carried out in the presence of an organic solvent.

13. Process according to claim 12, characterized in that the organic solvent is selected from the group ethers, amines, amides or mixtures thereof.

14. Process according to claim 11, characterized in that the compounds of formula (6) are di-tert-butyl dicarbonate.
